(19) United States
(12) Patent Application Publication
(10) Pub. No.: US 2012/0143042 A1
(43) Pub. Date: Jun. 7, 2012

(54) Ultrasound Methods, Systems and Computer Program Products for Imaging Fluids Using Acoustic Radiation Force

(76) Inventors: Mark L. Palmeri, Durham, NC (US); Samantha L. Lipman, Durham, NC (US); Kathryn R. Nightingale, Durham, NC (US)

(21) Appl. No.: 13/312,278

(22) Filed: Dec. 6, 2011

Related U.S. Application Data
(60) Provisional application No. 61/420,000, filed on Dec. 6, 2010.

(51) Int. Cl. A61B 8/00; A61B 6/00; A61B 5/055; A61B 8/08; A61B 8/14

(52) U.S. Cl. 600/411; 600/437; 600/443; 600/427

(57) ABSTRACT

The ultrasound system includes a controller configured to communicate with an ultrasound transducer such that the ultrasound transducer emits a radiation force excitation ultrasound pulse from the ultrasound transducer that propagates through the region of interest and is sufficient to perturb a fluid in the region of interest; emits a first and second acoustic ultrasound pulse from the ultrasound transducer that propagates away from the ultrasound transducer, through a region of interest and produces respective first and second echo ultrasound signal that propagate from the region of interest to the ultrasound transducer; and receives a first and second data set responsive to the first and second respective echo signals at the ultrasound transducer. A decorrelation module is configured to identify a decorrelation region of decorrelated data that is decorrelated between the first and second data sets, and the decorrelation region indicates a presence of fluid in the region of interest.

### RELATED APPLICATIONS

This applications claims priority to U.S. Provisional Application Ser. No. 61/420,000 filed Dec. 6, 2010, the disclosure of which is hereby incorporated by reference in its entirety.

### FIELD OF THE INVENTION

The present invention relates to ultrasound methods, systems and computer program products, and more specifically to ultrasound imaging of fluids.

### BACKGROUND

Fluids injected in to the body can be difficult to image using conventional techniques. Such fluids can be anechoic and diffuse throughout the adjacent soft tissue during injection, which can result in obscuration of the structures of interest in ultrasound imaging.

The accurate delineation of injected anesthetics during regional nerve blocks could ensure that adequate nerve blockages are achieved because regional nerve blocks ideally require a substantially even distribution of anesthetic around the circumference of a target nerve/plexus. Accurate feedback of the distribution of injected anesthetic during injection can allow the anesthesiologist to reposition the needle to achieve the desired distribution. Anesthetic drugs may be ineffective if the distribution of the drugs around a target nerve is insufficient, which may result in intraoperative interventions, reduced post-operative pain control, and reduced post-operative function.

U.S. Patent Publication No. 2010/0241001 discloses a cross correlation metrics that may be calculated between serial A-lines to quantify a degree of change. However, in slow injections or injections that involve small volumes, these changes may be small and difficult to detect relative to physiologic and physician-induced motion, which may also be a source of image decorrelation. The disclosure of U.S. Patent Publication No. 2010/0241001 is hereby incorporated by reference in its entirety.

### SUMMARY OF EMBODIMENTS OF THE INVENTION

In some embodiments, an ultrasound system for identifying a presence of fluid in a region of interest is provided. The system includes a controller configured to communicate with an ultrasound transducer such that the ultrasound transducer a) emits a radiation force excitation ultrasound pulse from the ultrasound transducer that propagates through the region of interest and is sufficient to perturb a fluid in the region of interest; b) emits a first acoustic ultrasound pulse from the ultrasound transducer that propagates away from the ultrasound transducer, through a region of interest and produces a first echo ultrasound signal that propagates from the region of interest to the ultrasound transducer; c) receives a first data set responsive to the first echo signal at the ultrasound transducer; d) emits a second acoustic ultrasound pulse from the ultrasound transducer that propagates away from the ultrasound transducer, through the region of interest and produces a second echo ultrasound signal that propagates from the region of interest to the ultrasound transducer while the radiation force excitation ultrasound pulse is perturbing the fluid in the region of interest; and e) receives a second data set responsive to the second echo signal at the ultrasound transducer. A decorrelation module is configured to identify a decorrelation region of decorrelated data that is decorrelated between the first and second data sets, and the decorrelation region indicates a presence of fluid in the region of interest.

In some embodiments, the region of interest corresponds to a plurality of location-defined pixels. The first data set includes a first plurality of signals that correspond to each of the plurality of location-defined pixels at a first time, the second data set includes a second plurality of signals that correspond to each of the plurality of location-defined pixels at a second time, and the decorrelation module is configured to compare respective ones of the first and second plurality of signals at corresponding ones of the plurality of location-defined pixels to identify decorrelated pixels, and the decorrelation region comprises the decorrelated pixels. The first time may be before the ultrasound transducer emits the radiation force excitation ultrasound pulse and the second time may be after the ultrasound transducer emits a radiation force excitation ultrasound pulse. The second time may be less than about 250 ms.

In some embodiments, the controller is further configured to control the ultrasound transducer so that the ultrasound transducer: f) emits one or more additional acoustic ultrasound pulses from the ultrasound transducer that propagate away from the ultrasound transducer, through the region of interest and produce corresponding additional echo ultrasound signal(s) that propagate from the region of interest to the ultrasound transducer; and g) receives one or more additional data sets responsive to the additional echo signals at the ultrasound transducer. The decorrelation module may be further configured to identify the decorrelation region of decorrelated data responsive to a magnitude of decorrelation between two or more of the first data set, the second data set and the additional data sets.

In some embodiments, the first time and second times are sequentially after the ultrasound transducer emits the radiation force excitation ultrasound pulse.

In some embodiments, the decorrelation module is configured to calculate a decorrelation magnitude of a difference between the respective ones of the first and second plurality of signals at the corresponding ones of the plurality of location-defined pixels. In some embodiments, the decorrelation module is configured to identify ones of the plurality of location-defined pixels as decorrelated pixels when the decorrelation magnitude is greater than a threshold value. In some embodiments, the decorrelation magnitude is based on a correlation coefficient (ρ) between a first signal, s₁(t), of the first plurality of signals at one of the plurality of location-defined pixels and a second signal, s₂(t), of the second plurality of radio frequency signals at one of the plurality of location-defined pixels as follows:

$$ρ = \frac{〈 {s}_{1} ( t ) {s}_{2}^{*} ( t ) 〉}{\sqrt{〈 {s}_{1} ( t ) {s}_{1}^{*} ( t ) 〉 〈 {s}_{2} ( t ) {s}_{2}^{*} ( t ) 〉}}$$

where * denotes the complex conjugate of the signal, ρ is a complex number whose magnitude denotes a similarity or correlation of the first and second signals and whose phase represents a phase difference between the first and second signals. In some embodiments, the decorrelation module is configured to identify a decorrelated pixel by applying a median filter to the correlation coefficient. In some embodiments, the decorrelation module identifies a decorrelation region of decorrelated pixels based on a decorrelation pattern having a central local minimum value indicating a relatively low correlation coefficient and an circumferential edge portion having higher correlation coefficients than the central local minimum value.

In some embodiments, the radiation force excitation ultrasound pulse has a duration of greater than 50 microseconds. In some embodiments, the radiation force excitation ultrasound pulse has a duration of greater than 400 microseconds. In some embodiments, the radiation force ultrasound pulse has a duration greater than 50 or 400 microseconds and less than 1 millisecond (1000 microseconds).

In some embodiments, the controller is configured to provide an image of the region of interest that identifies the decorrelation region.

In some embodiments, the fluid is a fluid injected into the region of interest. In some embodiments, the fluid is a fluid in a cyst.

In some embodiments, an ultrasound method for identifying a presence of fluid in a region of interest includes a) emitting a radiation force excitation ultrasound pulse from the ultrasound transducer that propagates through the region of interest and is sufficient to perturb a fluid in the region of interest; b) emitting a first acoustic ultrasound pulse from the ultrasound transducer that propagates away from the ultrasound transducer, through a region of interest and produces a first echo ultrasound signal that propagates from the region of interest to the ultrasound transducer; c) receiving a first data set responsive to the first echo signal at the ultrasound transducer; d) emitting a second acoustic ultrasound pulse from the ultrasound transducer that propagates away from the ultrasound transducer, through the region of interest and produces a second echo ultrasound signal that propagates from the region of interest to the ultrasound transducer while the radiation force excitation ultrasound pulse is perturbing the fluid in the region of interest; e) receiving a second data set responsive to the second echo signal at the ultrasound transducer; and f) identifying a decorrelation region of decorrelated data that is decorrelated between the first and second data sets, wherein the decorrelation region indicates a presence of fluid in the region of interest.

In some embodiments, a computer program product for identifying a presence of fluid in a region of interest includes a computer readable medium having computer readable program code embodied therein. The computer readable program code includes computer readable program code configured to emit a radiation force excitation ultrasound pulse from the ultrasound transducer that propagates through the region of interest and is sufficient to perturb a fluid in the region of interest; computer readable program code configured to emit a first acoustic ultrasound pulse from the ultrasound transducer that propagates away from the ultrasound transducer, through a region of interest and produces a first echo ultrasound signal that propagates from the region of interest to the ultrasound transducer; computer readable program code configured to receive a first data set responsive to the first echo signal at the ultrasound transducer; computer readable program code configured to emit a second acoustic ultrasound pulse from the ultrasound transducer that propagates away from the ultrasound transducer, through the region of interest and produces a second echo ultrasound signal that propagates from the region of interest to the ultrasound transducer while the radiation force excitation ultrasound pulse is perturbing the fluid in the region of interest; computer readable program code configured to receive a second data set responsive to the second echo signal at the ultrasound transducer; and computer readable program code configured to identify a decorrelation region of decorrelated data that is decorrelated between the first and second data sets, wherein the decorrelation region indicates a presence of fluid in the region of interest.

### BRIEF DESCRIPTION OF THE DRAWINGS

The accompanying drawings, which are incorporated in and constitute a part of the specification, illustrate embodiments of the invention and, together with the description, serve to explain principles of the invention.

FIG. 1 is a schematic drawing of an ultrasound system according to some embodiments of the present invention.

FIG. 2 is a flowchart illustrating operations according to embodiments of the present invention.

FIGS. 3A-3B are B-mode images above the rectus sheath in a cadaver before (FIG. 3A) and after (FIG. 3B) injection of 3 mL of saline. The superimposed highlighted regions indicate regions of decorrelation after the acoustic radiation force excitation, which are notably absent when no fluid was injected (FIG. 3A). The highlighted region on the right indicates increased decorrelation around the needle, consistent with the presence of the injectate in this region.

### DETAILED DESCRIPTION OF EMBODIMENTS OF THE INVENTION

The present invention now will be described hereinafter with reference to the accompanying drawings and examples, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

Like numbers refer to like elements throughout. In the figures, the thickness of certain lines, layers, components, elements or features may be exaggerated for clarity.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is inverted, elements described as “under” or “beneath” other elements or features would then be oriented “over” the other elements or features. Thus, the exemplary term “under” can encompass both an orientation of “over” and “under.” The device may be otherwise oriented (rotated 90 degrees or at other orientations) and the spatially relative descriptors used herein interpreted accordingly. Similarly, the terms “upwardly,” “downwardly,” “vertical,” “horizontal” and the like are used herein for the purpose of explanation only unless specifically indicated otherwise.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present invention. The sequence of operations (or steps) is not limited to the order presented in the claims or figures unless specifically indicated otherwise.

The present invention is described below with reference to block diagrams and/or flowchart illustrations of methods, apparatus (systems) and/or computer program products according to embodiments of the invention. It is understood that each block of the block diagrams and/or flowchart illustrations, and combinations of blocks in the block diagrams and/or flowchart illustrations, can be implemented by computer program instructions. These computer program instructions may be provided to a processor of a general purpose computer, special purpose computer, and/or other programmable data processing apparatus to produce a machine, such that the instructions, which execute via the processor of the computer and/or other programmable data processing apparatus, create means for implementing the functions/acts specified in the block diagrams and/or flowchart block or blocks.

These computer program instructions may also be stored in a computer-readable memory that can direct a computer or other programmable data processing apparatus to function in a particular manner, such that the instructions stored in the computer-readable memory produce an article of manufacture including instructions which implement the function/act specified in the block diagrams and/or flowchart block or blocks.

The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions/acts specified in the block diagrams and/or flowchart block or blocks.

Accordingly, the present invention may be embodied in hardware and/or in software (including firmware, resident software, micro-code, etc.). Furthermore, embodiments of the present invention may take the form of a computer program product on a computer-usable or computer-readable storage medium having computer-usable or computer-readable program code embodied in the medium for use by or in connection with an instruction execution system.

The computer-usable or computer-readable medium may be, for example but not limited to, an electronic, magnetic, optical, electromagnetic, infrared, or semiconductor system, apparatus or device. More specific examples (a non-exhaustive list) of the computer-readable medium would include the following: an electrical connection having one or more wires, a portable computer diskette, a random access memory (RAM), a read-only memory (ROM), an erasable programmable read-only memory (EPROM or Flash memory), an optical fiber, and a portable compact disc read-only memory (CD-ROM).

As used herein, the term “high intensity” or “radiation force excitation” refers to an ultrasonic or acoustic pulse having a Spatial Peak Temporal Average Intensity of sufficient strength (a desired combination of (i) amplitude, (ii) pulse length, and (iii) pulse repetition frequency), to initiate fluid movement or acoustic streaming. Acoustic streaming relies upon the radiation force phenomenon which is associated with all forms of wave motion. The radiation force phenomenon is caused by a transfer of momentum from a wave to absorbing and reflecting obstacles in its path. When a wave propagates through a fluid, this momentum transfer generates a bulk steady motion of the fluid in the direction of wave propagation.

The term “low intensity” refers to an ultrasonic or acoustic pulse having a Spatial Peak Temporal Average Intensity of insufficient strength (a desired combination of (i) amplitude, (ii) pulse length, and (iii) pulse repetition frequency), to initiate fluid movement or acoustic streaming in the target lesion.

The term “region” or “region of interest” refers generally to the area which is analyzed to detect the movement of any fluid existing within the lesion. The region interrogated by the present invention may include biological tissue such as animal tissue which may include lesion tissue. The present invention is not limited to biological systems, but may also be applied to other areas such as industrial applications, where the region maybe within the actual material being tested.

The term “differences” or “decorrelation” as used herein, in the context of the comparisons to be made between two or more reflected signals, refers any distinguishable feature or characteristic of the reflected signal that is quantifiable. Examples of differences which may be compared include, but are not limited to; the time of arrival of a signal, phase, amplitude, and the intensity of a signal.

The term “time of arrival” refers herein to the measured elapsed time between the transmission of a transmitting signal and the return of a corresponding reflected signal. The time of arrival is measured by conventional measurement techniques.

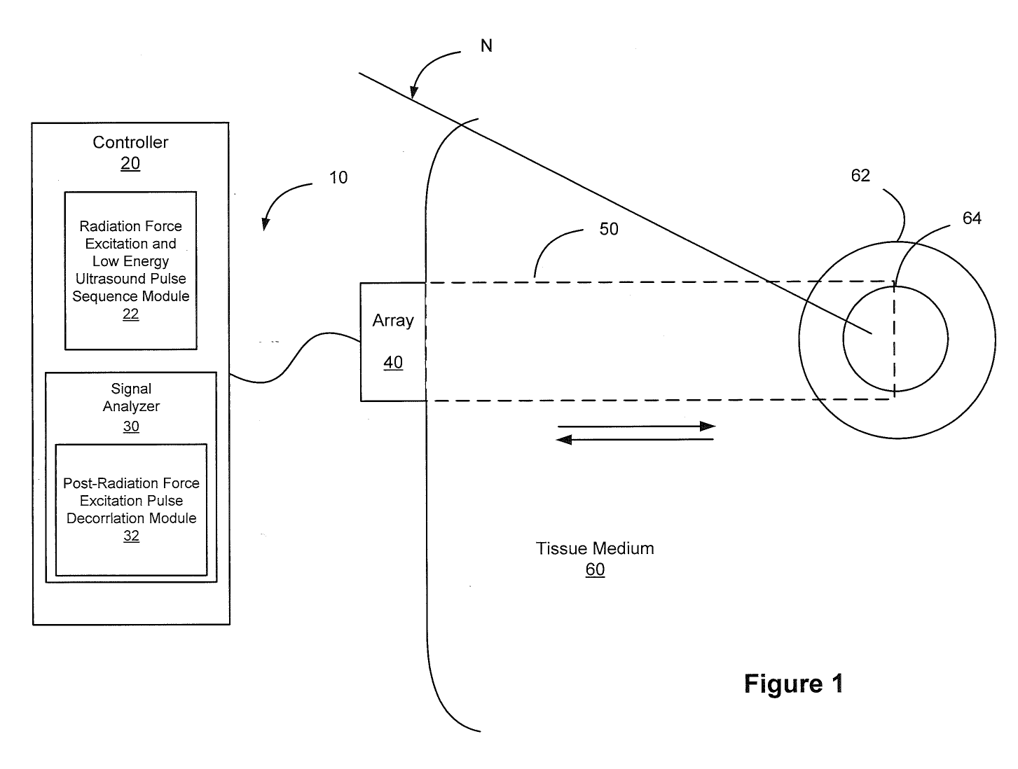

As illustrated in FIG. 1, an ultrasound system 10 includes a controller 20, a signal analyzer 30 and an ultrasound transducer array 40. The ultrasound transducer array 40 is configured to transmit and receive ultrasound signals 50, and may be contacted to a target medium such as a tissue medium 60. As illustrated, the tissue medium 60 includes a target region 62 having an injection region 64.

The controller 20 may include a radiation force excitation and low energy ultrasound pulse sequence module 22 that is configured to control the ultrasound array 40 to emit and receive ultrasound signals. For example, the pulse sequence module 22 may control the ultrasound array 40 to emit radiation force excitation acoustic that are sufficient to initiate fluid movement and/or acoustic streaming in the target region 62, and low energy ultrasound pulses that are configured to produce an echo pulse in the region of interest that may be received by the ultrasound array 40 and/or analyzed as radio-frequency (RF) data by the signal analyzer 30.

The signal analyzer 30 may include a post-radiation force excitation pulse decorrelation module 32. In some embodiments, the post-radiation force excitation pulse decorrelation module is configured to compare ultrasound data received at the ultrasound array 40 after a radiation force excitation pulse is delivered to the target region 62 to identify regions of decorrelated data or data that is sufficiently decorrelated to indicate a change due to the presence of fluid in the tissue. The ultrasound data being compared may include a reference signal that is obtained either before or after the radiation force excitation pulse. The ultrasound data may include radio-frequency ultrasound echo signal that results from a low-energy acoustic ultrasound pulse emitted in the target region 62. Without wishing to be bound by any particular theory, it is currently believed that the tissue movement from a radiation force excitation may increase a decorrelation of data due to fluid flowing from the needle N and injected into the tissue medium 60. Thus, the magnitude of decorrelation of ultrasound data before the radiation force excitation pulse and after the radiation force excitation may be increased for regions of tissue in which an injected fluid from the needle N is present. A magnitude of decorrelation may be measured by a decorrelation coefficient as described herein and generally refers to an amount or degree of difference between two signals. In some embodiments, the decorrelated data includes radiofrequency ultrasound data obtained from an echo signal that is responsive to a low energy acoustic ultrasound signal applied to the tissue, such as in B-mode imaging.

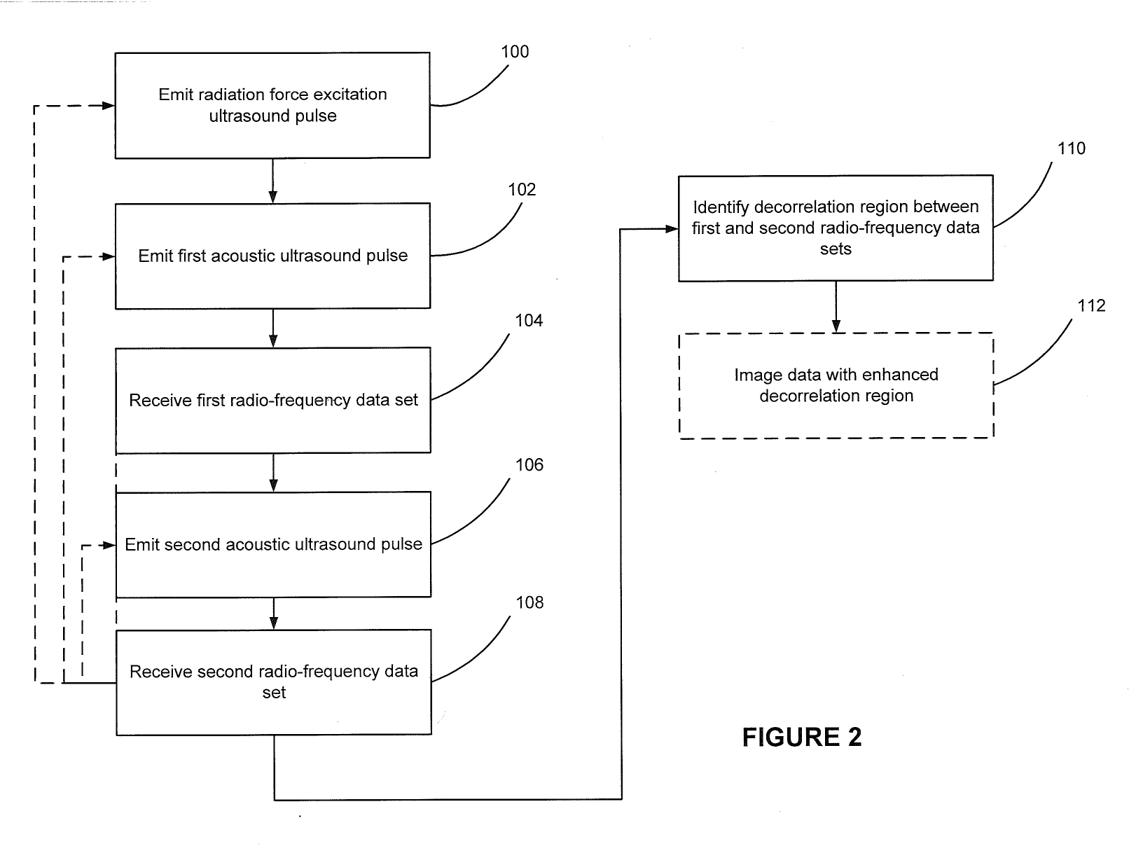

For example, with reference to FIGS. 1 and 2, the radiation force excitation and low energy ultrasound pulse sequence module 22 controls the array 40 to emit a radiation force excitation ultrasound pulse that is sufficient to perturb an injected fluid in the target region 62 (Block 100). The radiation force excitation and low energy ultrasound pulse sequence module 22 may control the ultrasound transducer array 40 to emit a first acoustic ultrasound pulse (Block 102), which propagates away from the array 40, through the tissue medium 60, and is reflected at the target region 62. The resulting echo signal propagates back to the array 40, which receives the echo signal as a first radio-frequency data set (Block 104). The first radio-frequency data set may be referred to as a “reference” data set or signal. The pulse sequence module 22 may then control the array 40 to emit a second acoustic ultrasound pulse that propagates through the tissue medium 60, through the target region 62, where it is reflected as an echo signal (Block 106). The echo signal is received by the array 40 as a second radio frequency data set (Block 108). It should be understood that Blocks 100-108 may be performed during the injection of a fluid into the injection region 64 of the tissue 60. The post-radiation force excitation pulse decorrelation module 32 may identify a decorrelation region in the injection region 64 of the tissue that indicates an increased likelihood of the presence of the injected fluid (Block 110),

After the delivery of the radiation force, excitation, the tissue in the target region 62 may be sufficiently perturbed so that any injected fluid may be detected or identified by the decorrelation module 32 for a period of time or “perturbation time,” Accordingly, the second ultrasound pulse may be delivered (Block 106) and the second resulting echo radio-frequency data set may be received (Block 108) during the perturbation time period after the delivery of the radiation force excitation. In some embodiments, the perturbation time period is less than about 250 milliseconds. In particular embodiments, data sets obtained after the perturbation time period are excluded. Without wishing to be bound by any particular theory, it is currently believed that the decorrelation sensitivity may be increased by comparing radio-frequency data sets obtained during the perturbation time after the delivery of a radiation force excitation. The fluids, which are not physically tethered to the tissue, may move in such a way that the decorrelation is greater over a longer time domain than the soft tissue, The radiation force excitation ultrasound pulse may be greater than that typically used in ARFI imaging, such as an ultrasound pulse having a duration of greater than about 50 microseconds or greater than about 400 microseconds and up to about 1 millisecond.

In some embodiments, the second acoustic ultrasound pulse may be delivered (106) and the second radio-frequency data set may be received (Block 108) repeatedly after the radiation force excitation pulse (Block 104) to provide additional sets of data that may be compared with the first radio-frequency data set (Block 104), the second radio-frequency data set (Block 108) and/or additional ones of the sets of radio-frequency data. Moreover, it should be understood that the operational blocks in FIG. 2 are not necessarily performed in the order shown in FIG. 2. For example, the radiation force excitation ultrasound pulse may be emitted (Block 100) after the first acoustic ultrasound pulse is emitted (Block 102) and/or after the first radio-frequency data set is received (Block 104) such that a reference radio-frequency data set is obtained before the emission of the radiation force excitation ultrasound pulse. It should be further understood that the operations shown in FIG. 2 may be repeated sequentially and/or in different orders to obtain data sets before and/or after the emission of the radiation force excitation ultrasound pulse and any two or more radio-frequency data sets may be compared as described herein to identify an increased likelihood of the presences of the injected fluid (Block 110). At least one of the data sets is obtained during the perturbation time period, e.g., about 250 milliseconds after the acoustic radiation force excitation. In some embodiments, more than two radio-frequency data sets may be compared to determine a decorrelation region, and a cumulative decorrelation region may be identified based on the comparisons. The data image that identifies the decorrelation region may identify the cumulative decorrelation region to indicate where fluid has likely been injected, e.g., cumulatively over a period of time. The visual identification of the decorrelation region may be by using color, contrast, lines, etc. that may be superimposed on any suitable medical image, such as a B-Mode and/or ARFI image. Although embodiments according to the present invention are described herein with respect to decorrelated ultrasound signals, it should be understood that regions of decorrelation induced by acoustic radiation force perturbation of fluids may also be detected and/or imaged by non-ultrasound imaging systems, such as magnetic resonance imaging or optical coherence tomography by using non-ultrasound imaging signals.

In some embodiments, the controller 20 is configured to provide an image of the tissue medium 60 that identifies the decorrelation region (FIG. 2, Block 112). For example, an image can be provided in which the decorrelation region is shown in a contrasting color. In some embodiments, the ultrasound images described herein can be used to provide substantially real-time feedback during injection procedures. The image can include a target nerve and an approximation of the distribution of injected fluid, such as an anesthetic, substantially in real-time during injection. A health care professional can view the image to ensure that a desired distribution of anesthetic is achieved around the targeted nerve. In some embodiments, the health care professional can reposition the needle to achieve the desired distribution. Accordingly, embodiments of the present invention can be used to reduce incidences of failed nerve blocks.

In some embodiments, the target region 62 corresponds to a plurality of location-defined pixels. The first and second radio-frequency data sets include respective radio-frequency signal that correspond to each of the plurality of location-defined pixels. The decorrelation module 32 is configured to compare respective radio-frequency signals from the first and second data sets at the location-defined pixels to identify decorrelated pixels. The decorrelation module 32 identifies the decorrelation region based on the identified decorrelated pixels.

The decorrelation module 32 is configured to identify ones of the plurality of location-defined pixels as decorrelated pixels when a decorrelation magnitude of a pixel is greater than a threshold value. The decorrelation magnitude may be based on a correlation coefficient (ρ) between a first signal, s₁(t), of the first plurality of radio-frequency signals at one of the plurality of location-defined pixels and a second signal, s₂(t), of the second plurality of radio frequency signals at one of the plurality of location-defined pixels as follows:

$$ρ = \frac{〈 {s}_{1} ( t ) {s}_{2}^{*} ( t ) 〉}{\sqrt{〈 {s}_{1} ( t ) {s}_{1}^{*} ( t ) 〉 〈 {s}_{2} ( t ) {s}_{2}^{*} ( t ) 〉}}$$

where * denotes the complex conjugate of the signal, ρ is a complex number whose magnitude denotes a similarity or correlation of the first and second signals and whose phase represents a phase difference between the first and second signals. The decorrelation module may be configured to identify a decorrelated pixel by applying a median filter to the correlation coefficient using adjacent pixel correlation coefficients.

In some embodiments, the decorrelation module 32 may identify a decorrelation region of decorrelated pixels based on a decorrelation pattern having a central local minimum value indicating a relatively low correlation coefficient and an circumferential edge portion having higher correlation coefficients than the central local minimum value. For example, correlation coefficients for the pixels for a frame or data set at a given time may be smoothed using smoothing function such as a median filter. The decorrelation module 32 may identify the lowest correlation coefficient in the region of interest. It is currently believed that the injected fluids may form a pocket or sub-region with higher correlation coefficients in the region surrounding the injection site. The decorrelation module 32 may analyze a lateral line passing through an identified lowest correlation coefficient (or highest decorrelation value), and if the region is a decorrelated region indicating an increased likelihood of an injected fluid, then a three-dimensional graph of the correlation coefficients of pixels in the region may be shaped like a valley with a peak on either side. To identify the decorrelation region, the decorrelation module 32 calculates the correlation coefficient as a minimum correlation coefficient plus a fraction (e.g., two-thirds, or ranging from a minimum of zero (i.e., no correlation) to 1 (perfect correlation)) of the difference between the highest correlation coefficient and the lowest correlation coefficient. For example, if the lowest correlation coefficient is 0.5 with peaks of 0.8 on either side, then the decorrelation module 32 would set a threshold of 0.7 for that time-specific data set or frame to identify a pixel as part of a decorrelation region.

Although embodiments of the current invention are described herein with respect to injected fluids for use in anesthetic procedures, it should be understood that embodiments of the current invention can be applied to other procedures using injected fluids, such as amniotic fluid injections in obstetrics, corticosteroid injection in orthopedic surgery and/or sports medicine, the delivery of drugs (e.g., chemotherapeutic drug injection into tumors) and fluid removal in fine needle aspirations (FNAs). In some embodiments, the fluid is not an injected fluid. Embodiments of the current invention may be used to distinguish a fluid-rich region from a region without fluid. For example, embodiments of the current invention may be used to distinguish between fluid-filled cysts and solid masses.

Moreover, embodiments of the current invention can be used with conventional B-mode ultrasound imaging data and/or acoustic radiation force imaging (ARFI) data. For example, the controller 20 and ultrasound array 40 can be configured to obtain conventional B-mode images or ARFI images in which the array 40 emits a series of low intensity “tracking lines” and higher intensity “pushing” pulses to interrogate the tissue medium 60. Various ultrasound techniques are described, for example, in U.S. Pat. Nos. 7,374,538 and 6,371,912, the disclosures of which are hereby incorporated by reference in their entireties. In some embodiments, B-mode and ARFI imaging data can be combined to provide a single image, and the decorrelation region or decorrelation map can be identified on the combined image. Moreover, two- or three-dimensional images can be used. It should also be understood that the ultrasound array 40 can be a one- or two-dimensional array having various numbers of ultrasound array elements.

In some embodiments, an image may be provided during an injection procedure in which the injected anesthetic is highlighted relative to the nerve position based on a detected decorrelation region as discussed herein. The medical health professional may then reposition the needle for a more effective nerve block based on the distribution. More efficacious nerve blocks may improve post-operative pain management and reduce the rescue interventions that may be needed if a block fails intraoperatively (e.g., rescue block or conversion to general anesthesia), which may reduce pain medication and the costs associated with rescue interventions. The volume of injected anesthetic may be reduced and confidence in its distribution may be improved. Potential nerve and cardiac toxicity that can occur with large volumes of injected anesthetics may be reduced.

Embodiments according to the present invention will now be described with respect to the following non-limiting examples.

### EXAMPLE

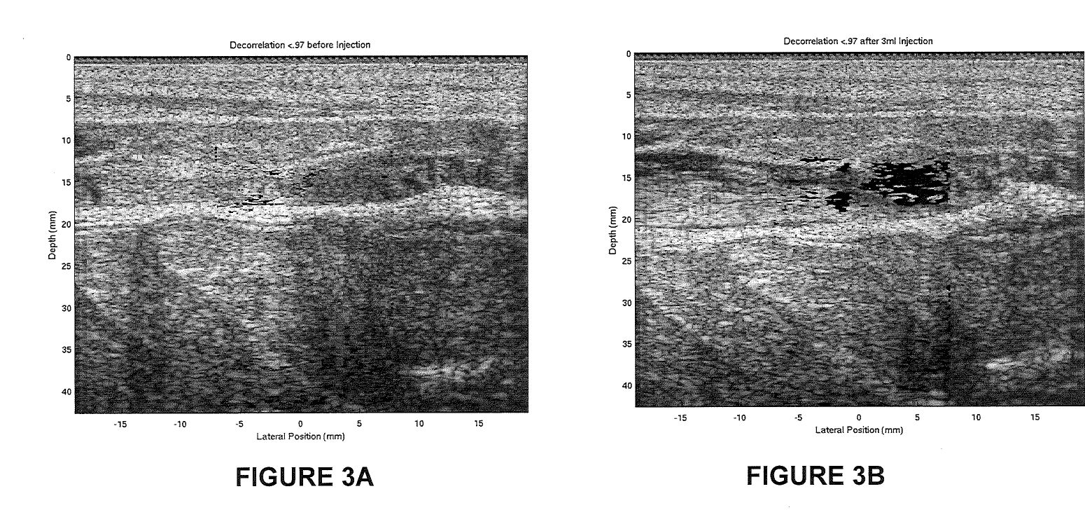

The feasibility of using high-intensity ultrasonic radiation force excitation during the slow injection to perturb the fluid preferentially so that the decorrelations are strong enough to be distinguished from other sources of motion in the image was demonstrated in a cadaveric experiment in three different imaging sites: brachial plexus, free nerve endings in the rectus sheath, and the femoral nerve; all of which are common targets for peripheral nerve blocks. FIG. 3 illustrates a representative image with and without an injection with an acoustic radiation force excitation using the Siemens® VF7-3 linear array.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

That which is claimed is:

1. An ultrasound system for identifying a presence of fluid in a region of interest, the system comprising:
a controller configured to communicate with an ultrasound transducer such that the ultrasound transducer
a) emits a radiation force excitation ultrasound pulse from the ultrasound transducer that propagates through the region of interest and is sufficient to perturb a fluid in the region of interest;
b) emits a first acoustic ultrasound pulse from the ultrasound transducer that propagates away from the ultrasound transducer, through the region of interest and produces a first echo ultrasound signal that propagates from the region of interest to the ultrasound transducer;
c) receives a first data set responsive to the first echo signal at the ultrasound transducer;
d) emits a second acoustic ultrasound pulse from the ultrasound transducer that propagates away from the ultrasound transducer, through the region of interest and produces a second echo ultrasound signal that propagates from the region of interest to the ultrasound transducer while the radiation force excitation ultrasound pulse is perturbing the fluid in the region of interest; and
e) receives a second data set responsive to the second echo signal at the ultrasound transducer; and
a decorrelation module configured to identify a decorrelation region of decorrelated data that is decorrelated between the first and second data sets, wherein the decorrelation region indicates a presence of fluid in the region of interest.

2. The ultrasound system of claim 1, wherein the region of interest corresponds to a plurality of location-defined pixels, and the first data set includes a first plurality of signals that correspond to each of the plurality of location-defined pixels at a first time, and the second data set includes a second plurality of signals that correspond to each of the plurality of location-defined pixels at a second time, and the decorrelation module is configured to compare respective ones of the first and second plurality of signals at corresponding ones of the plurality of location-defined pixels to identify decorrelated pixels, and the decorrelation region comprises the decorrelated pixels.

3. The ultrasound system of claim 2, wherein the first time is before the ultrasound transducer emits the radiation force excitation ultrasound pulse and the second time is after the ultrasound transducer emits a radiation force excitation ultrasound pulse.

4. The ultrasound system of claim 3, wherein the second time is less than about 250 ms.

5. The ultrasound system of claim 3, wherein the controller is further configured to control the ultrasound transducer so that the ultrasound transducer:
f) emits one or more additional acoustic ultrasound pulses from the ultrasound transducer that propagate away from the ultrasound transducer, through the region of interest and produce corresponding additional echo ultrasound signal(s) that propagate from the region of interest to the ultrasound transducer; and
g) receives one or more additional data sets responsive to the additional echo signals at the ultrasound transducer;
wherein the decorrelation module is further configured to identify the decorrelation region of decorrelated data responsive to a magnitude of decorrelation between two or more of the first data set, the second data set and the additional data sets.

6. The ultrasound system of claim 2, wherein the first time and second times are sequentially after the ultrasound transducer emits the radiation force excitation ultrasound pulse, and the second time is less than about 250 ms.

7. The ultrasound system of claim 2, wherein the decorrelation module is configured to calculate a decorrelation magnitude of a difference between the respective ones of the first and second plurality of signals at the corresponding ones of the plurality of location-defined pixels.

8. The ultrasound system of claim 7, wherein the decorrelation module is identify ones of the plurality of location-defined pixels as decorrelated pixels when the decorrelation magnitude is greater than a threshold value.

9. The ultrasound system of claim 7, wherein the decorrelation magnitude is based on a correlation coefficient (ρ) between a first signal, s₁(t), of the first plurality of signals at one of the plurality of location-defined pixels and a second signal, s₂(t), of the second plurality of radio frequency signals at one of the plurality of location-defined pixels as follows:
$$ρ = \frac{〈 {s}_{1} ( t ) {s}_{2}^{*} ( t ) 〉}{\sqrt{〈 {s}_{1} ( t ) {s}_{1}^{*} ( t ) 〉 〈 {s}_{2} ( t ) {s}_{2}^{*} ( t ) 〉}}$$
where * denotes the complex conjugate of the signal, ρ is a complex number whose magnitude denotes a similarity or correlation of the first and second signals and whose phase represents a phase difference between the first and second signals.

10. The ultrasound system of claim 9, wherein the decorrelation module is configured to identify a decorrelated pixel by applying a median filter to the correlation coefficient.

11. The ultrasound system of claim 9, wherein the decorrelation module identifies a decorrelation region of decorrelated pixels based on a decorrelation pattern having a central local minimum value indicating a relatively low correlation coefficient and an circumferential edge portion having higher correlation coefficients than the central local minimum value.

12. The ultrasound system of claim 1, wherein the radiation force excitation ultrasound pulse has a duration of greater than 50 microseconds.

13. The ultrasound system of claim 1, wherein the radiation force excitation ultrasound pulse has a duration of greater than 400 microseconds.

14. The ultrasound system of claim 1, wherein the controller is configured to provide an image of the region of interest that identifies the decorrelation region.

15. The ultrasound system of claim 1, wherein the fluid is a fluid injected into the region of interest.

16. The ultrasound system of claim 1, wherein the fluid is a fluid in a cyst.

17. An ultrasound method for identifying a presence of fluid in a region of interest, the method comprising:
a) emitting a radiation force excitation ultrasound pulse from the ultrasound transducer that propagates through the region of interest and is sufficient to perturb a fluid in the region of interest;
b) emitting a first acoustic ultrasound pulse from the ultrasound transducer that propagates away from the ultrasound transducer, through the region of interest and produces a first echo ultrasound signal that propagates from the region of interest to the ultrasound transducer;
c) receiving a first data set responsive to the first echo signal at the ultrasound transducer;
d) emitting a second acoustic ultrasound pulse from the ultrasound transducer that propagates away from the ultrasound transducer, through the region of interest and produces a second echo ultrasound signal that propagates from the region of interest to the ultrasound transducer while the radiation force excitation ultrasound pulse is perturbing the fluid in the region of interest;
e) receiving a second data set responsive to the second echo signal at the ultrasound transducer; and
f) identifying a decorrelation region of decorrelated data that is decorrelated between the first and second data sets, wherein the decorrelation region indicates a presence of fluid in the region of interest.

18. The method of claim 17, wherein the region of interest corresponds to a plurality of location-defined pixels, and the first data set includes a first plurality of signals that correspond to each of the plurality of location-defined pixels at a first time, and the second data set includes a second plurality of signals that correspond to each of the plurality of location-defined pixels at a second time, and the decorrelation module is configured to compare respective ones of the first and second plurality of signals at corresponding ones of the plurality of location-defined pixels to identify decorrelated pixels, and the decorrelation region comprises the decorrelated pixels.

19. The method of claim 18, wherein the first time is before the ultrasound transducer emits the radiation force excitation ultrasound pulse and the second time is after the ultrasound transducer emits a radiation force excitation ultrasound pulse.

20. The method of claim 19, wherein the second time is less than about 250 ms.

21. The method of claim 19, wherein the controller is further configured to control the ultrasound transducer so that the ultrasound transducer:
f) emits one or more additional acoustic ultrasound pulses from the ultrasound transducer that propagate away from the ultrasound transducer, through the region of interest and produce corresponding additional echo ultrasound signal(s) that propagate from the region of interest to the ultrasound transducer; and
g) receives one or more additional data sets responsive to the additional echo signals at the ultrasound transducer;
wherein the decorrelation module is further configured to identify the decorrelation region of decorrelated data responsive to a magnitude of decorrelation between two or more of the first data set, the second data set and the additional data sets.

22. The method of claim 18, wherein the first time and second times are sequentially after the ultrasound transducer emits the radiation force excitation ultrasound pulse, and the second time is less than about 250 ms.

23. The method of claim 22, wherein the decorrelation magnitude is based on a correlation coefficient (ρ) between a first signal, s₁(t), of the first plurality of signals at one of the plurality of location-defined pixels and a second signal, s₂(t), of the second plurality of radio frequency signals at one of the plurality of location-defined pixels as follows:
$$ρ = \frac{〈 {s}_{1} ( t ) {s}_{2}^{*} ( t ) 〉}{\sqrt{〈 {s}_{1} ( t ) {s}_{1}^{*} ( t ) 〉 〈 {s}_{2} ( t ) {s}_{2}^{*} ( t ) 〉}}$$
where * denotes the complex conjugate of the signal, ρ is a complex number whose magnitude denotes a similarity or correlation of the first and second signals and whose phase represents a phase difference between the first and second signals.

24. The method of claim 23, wherein the decorrelation module is configured to identify a decorrelated pixel by applying a median filter to the correlation coefficient.

25. The method of claim 23, wherein the decorrelation module identifies a decorrelation region of decorrelated pixels based on a decorrelation pattern having a central local minimum value indicating a relatively low correlation coefficient and an circumferential edge portion having higher correlation coefficients than the central local minimum value.

26. A computer program product for identifying a presence of fluid in a region of interest comprising: a computer readable medium having computer readable program code embodied therein, the computer readable program code comprising:
computer readable program code configured to emit a radiation force excitation ultrasound pulse from the ultrasound transducer that propagates through the region of interest and is sufficient to perturb a fluid in the region of interest;
computer readable program code configured to emit a first acoustic ultrasound pulse from the ultrasound transducer that propagates away from the ultrasound transducer, through the region of interest and produces a first echo ultrasound signal that propagates from the region of interest to the ultrasound transducer;
computer readable program code configured to receive a first data set responsive to the first echo signal at the ultrasound transducer;
computer readable program code configured to emit a second acoustic ultrasound pulse from the ultrasound transducer that propagates away from the ultrasound transducer, through the region of interest and produces a second echo ultrasound signal that propagates from the region of interest to the ultrasound transducer while the radiation force excitation ultrasound pulse is perturbing the fluid in the region of interest;
computer readable program code configured to receive a second data set responsive to the second echo signal at the ultrasound transducer; and
computer readable program code configured to identify a decorrelation region of decorrelated data that is decorrelated between the first and second data sets, wherein the decorrelation region indicates a presence of fluid in the region of interest.

27. An system for identifying a presence of fluid in a region of interest, the system comprising:
a controller configured to communicate with an ultrasound transducer such that the ultrasound transducer emits a radiation force excitation ultrasound pulse from the ultrasound transducer that propagates through the region of interest and is sufficient to perturb a fluid in the region of interest;
wherein the controller is further configured to communicate with an imaging apparatus such that the imaging apparatus emits a first imaging pulse from the imaging apparatus that propagates away from the imaging apparatus, through the region of interest and produces a first response signal that is received by the imaging apparatus as a first data set; emits a second imaging pulse from the ultrasound transducer that propagates away from the imaging apparatus, through the region of interest and produces a second response signal that is received by the imaging apparatus while the radiation force excitation ultrasound pulse is perturbing the fluid in the region of interest; and
a decorrelation module configured to identify a decorrelation region of decorrelated data that is decorrelated between the first and second data sets, wherein the decorrelation region indicates a presence of fluid in the region of interest.

28. The system of claim 27, wherein the imaging device is different from the ultrasound transducer.

29. The system of claim 27, wherein the imaging device is a magnetic resonance imaging device or an optical coherency tomography imaging device.
